(19) United States
(12) Patent Application Publication
(10) Pub. No.: US 2022/0001072 A1
(43) Pub. Date: Jan. 6, 2022

(54) AIR CONDITIONER WITH BIPOLAR IONIZATION ASSEMBLY

(71) Applicant: Matt Philip Davidson, St Petersburg, FL (US)

(72) Inventor: Matt Philip Davidson, St Petersburg, FL (US)

(21) Appl. No.: 17/305,281

(22) Filed: Jul. 2, 2021

Related U.S. Application Data
(60) Provisional application No. 62/705,537, filed on Jul. 2, 2020.

(51) Int. Cl. A61L 9/22; F24F 1/02; F24F 1/0353; F24F 1/04; F24F 8/30; F24F 8/80

(52) U.S. Cl. CPC A61L 9/22; F24F 1/02; F24F 1/0353; F24F 2110/66; F24F 8/30; F24F 8/80; F24F 1/04

(57) ABSTRACT

An air treatment system comprises an air conditioner; a bipolar ionization assembly; and a power supply. The air conditioner may portable and include a wheeled housing; an evaporator or entry grill for ambient air; a condenser grill for hot air discharge; a cold air supply discharge grate; a motor; an evaporator coil; and an evaporator blower intake. The bipolar ionization assembly includes electrical leads, and is mounted by a bracket to the evaporator blower intake to position the electrical leads within the air stream of the evaporator blower intake. The power supply is operably connected to the portable air conditioner and to the bipolar ionization assembly. When mounted in the air conditioner, the bipolar ionization assembly creates an antimicrobial plasma capable of killing harmful pathogens including bacteria, mycoplasma, viruses, mold and odors, and reducing the presence of Volatile Organic Compounds (VOC's), particulates, and static electricity in the air.

### CROSS-REFERENCE TO RELATED APPLICATION

This application claims the benefit of priority of U.S. Provisional Application No. 62/705,537, filed Jul. 2, 2020, the contents of which are herein incorporated by reference.

### BACKGROUND OF THE INVENTION

The present invention relates to air purification, and more particularly to an air conditioner with a bipolar ionization assembly. Even more particularly the present invention relates to a commercial portable air conditioner with a bipolar ionization assembly.

It is commonly known that infectious diseases, including dangerous pathogens like viruses and bacteria are communicated through both airborne means and human touch or contact. Airborne transmission spread occurs by an infected person coughing, sneezing, or vomiting, as microscopic aerosol droplets are expelled which may travel through a space where another person makes direct contact with these droplets and becomes infected. Further, in surface to person spread, an infected person may touch a surface, which another person may touch and become infected. Therefore, surfaces including work areas, desktops, tables, doorknobs, handles, keyboards, and equipment can spread these dangerous pathogens and diseases.

The commercial and industrial industries, and particularly, the healthcare and medical industries have utilized portable air conditioners for decades to provide short term cooling issues and emergency cooling applications within their facilities. These facilities include hospitals, health & rehabilitation facilities and medical labs. Portable air conditioners are used in various areas including emergency rooms, patient areas, nursing stations and other areas needing immediate climate control. Currently, such portable air conditioners do not provide adequate filtration and distribute dirty air containing, viruses, bacteria, mold spores, fungi, pet dander and a host of other dangerous particulate into the air, distributing it throughout a facility. As the healthcare and medical industries have no choice but to use portable air conditioning units in their facilities when needed, they are particularly concerned with indoor air quality (IAQ) and possible infection transmission to patients and staff. Further, it is unavoidable to breathe in contaminated air or touch various surfaces within the facilities that could possibly transmit these dangerous pathogens to healthcare workers or patients.

As can be seen, there is a need for a solution to the aforementioned problems.

### SUMMARY OF THE INVENTION

Advantageously, an embodiment of the present invention provides an air conditioner with a bipolar ionization assembly which solves the aforementioned problems. In one aspect the present invention provides a commercial portable air conditioner with a bipolar ionization assembly.

In one aspect, the present invention provides an air treatment system comprising an air conditioner; a bipolar ionization assembly; and a power supply. The air conditioner may portable and include a wheeled housing; an evaporator or entry grill for ambient air; a condenser grill for hot air discharge; a cold air supply discharge grate; a motor; an evaporator coil; and an evaporator blower intake. The bipolar ionization assembly includes electrical leads, and is mounted by a bracket to the evaporator blower intake to position the electrical leads within the air stream of the evaporator blower intake. The power supply is operably connected to the portable air conditioner and to the bipolar ionization assembly. When mounted in the air conditioner, the bipolar ionization assembly creates an antimicrobial plasma capable of killing harmful pathogens including bacteria, mycoplasma, viruses, mold and odors, and reducing the presence of Volatile Organic Compounds (VOC's), particulates, and static electricity in the air.

In one aspect the commercial portable air conditioner with a bipolar ionization assembly of the present invention is capable of killing contaminants within a space on contact and is more effective than HEPA and Ultra Violet light (UV). The present invention is capable of killing viruses, mold spores, and bacteria both airborne and on surfaces, and eliminates odors at their source while providing cooling in a safe and efficient manner. The commercial portable air conditioner with a bipolar Ionization assembly and combination of cooling and sanitizing the air is safer, more effective and more efficient than any available commercial portable air conditioner on the market.

These and other features, aspects and advantages of the present invention will become better understood with reference to the following drawings, description and claims.

### BRIEF DESCRIPTION OF THE DRAWINGS

The preferred embodiment of the claimed subject matter will hereinafter be described in conjunction with the appended drawings provided to illustrate and not to limit the scope of the claimed subject matter, where like designations denote like elements, and in which:

FIG. 1 is a perspective view of an exemplary embodiment of the present invention;

FIG. 2 is an exploded view of an exemplary embodiment of the present invention;

FIG. 3 is a perspective view of an exemplary embodiment of the present invention;

FIG. 4 is an exploded schematic view of an exemplary embodiment of the present invention; and

FIG. 5 is a perspective view of an exemplary embodiment of the present invention.

It is to be understood that like reference numerals refer to like parts throughout the several views of the drawings.

### DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

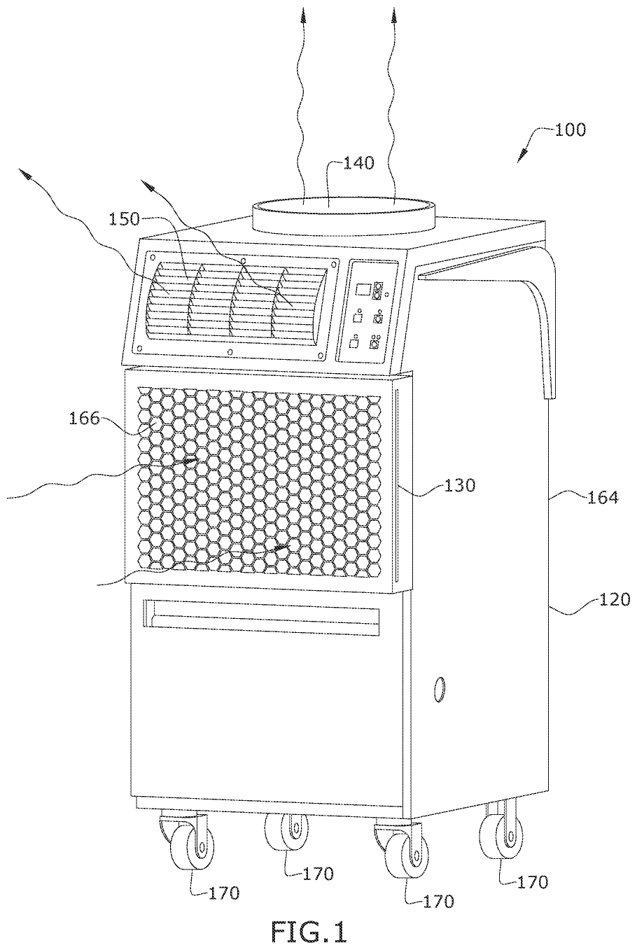

As used herein, “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, as there is no intention to be bound by any express or implied theory presented in the preceding technical field, background, brief summary or the following detailed description, it is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Broadly, an embodiment of the present invention provides an air conditioner with a bipolar ionization assembly which solves the aforementioned problems. In an exemplary embodiment, an air treatment system may include a portable air conditioner with a bipolar ionization assembly, and a power supply. The commercial portable air conditioner with a bipolar ionization assembly of the present invention is more effective than HEPA filters and UV light at eliminating pathogens, bacterial, viruses and mold spores. The bipolar ionization assembly of the present invention kills contaminants within a space on contact and is more effective than HEPA filters in reducing active microorganisms. A HEPA filter only traps particles down to 0.03 microns in size. Microbes smaller than 0.03 microns, which includes many viruses like Coronavirus, will pass directly through the HEPA filter and be recirculated into the space. Furthermore, a HEPA filter can only trap contaminants, and cannot treat the source of the contaminants. HEPA filters are not only ineffective on odors, gases and volatile organic compounds (VOCs), but also restrict airflow of the commercial portable air conditioner, making it less efficient on the cooling system. HEPA filters need to be replaced regularly and are expensive. The inventive commercial portable air conditioner with bipolar ionization assembly is maintenance free and requires no filters, thereby eliminating the cost and downtime associated with filter replacements.

Similarly, Ultra Violet light (UV) used in commercial portable air conditioners is limited in effectiveness. The effectiveness of UV light disinfection of air is dependent upon contaminants being exposed to the light long enough for the contaminants to be treated. Most commercial portable air conditioners move the airstream at 400 cfm allowing the contaminant to only be exposed to UV for a fraction of a second, which is ineffective in killing contaminants such as bacteria and viruses which need to be exposed to UV light between 15-30 minutes for effective treatment. Furthermore, UV light is ineffective and does not reduce airborne particulates, odors, gases and VOCs from the air. Costs are also an issue and include yearly maintenance and costly replacement of the UV bulbs.

The inventive commercial portable air conditioner with a bipolar ionization assembly in accordance with the present invention distinguishes over existing commercial portable air conditioners using HEPA filters and UV light for contaminant removal, which are not only substandard, but also inefficient and ineffective for complete microbe and odor removal from air and surfaces. The present invention kills viruses, mold spores, and bacteria both airborne and on surfaces. It eliminates odors at their source while providing cooling in a safe and efficient manner. The commercial portable air conditioner with a bipolar Ionization assembly and combination of cooling and sanitizing the air is safer, more effective and more efficient than any available commercial portable air conditioner on the market.

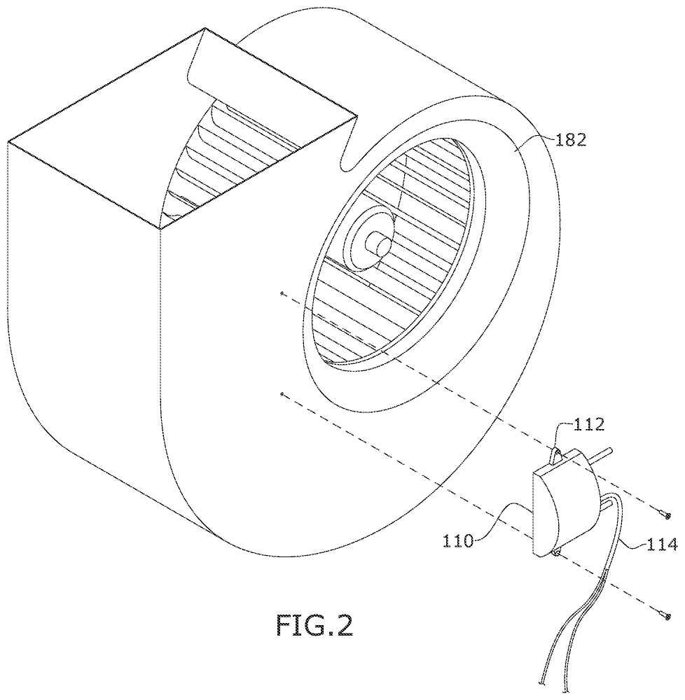

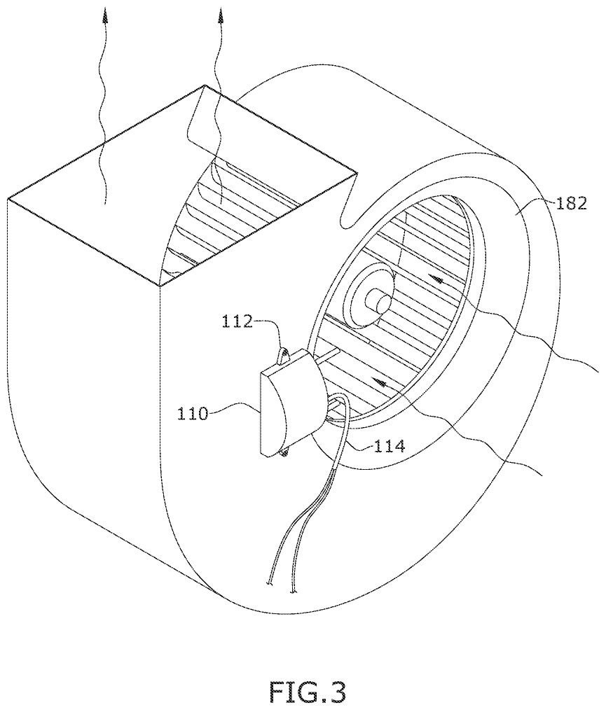

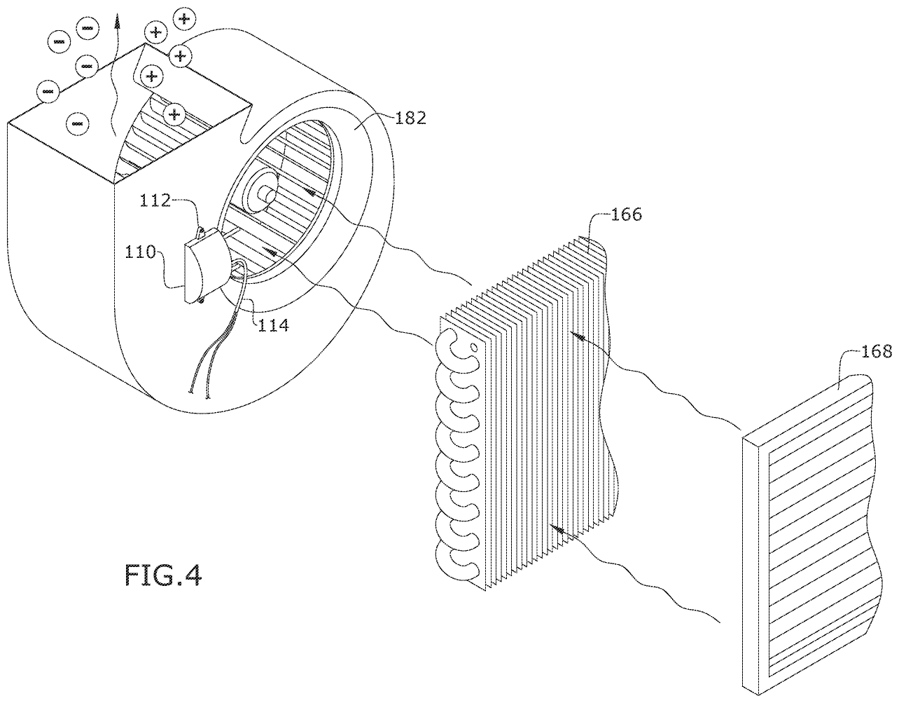

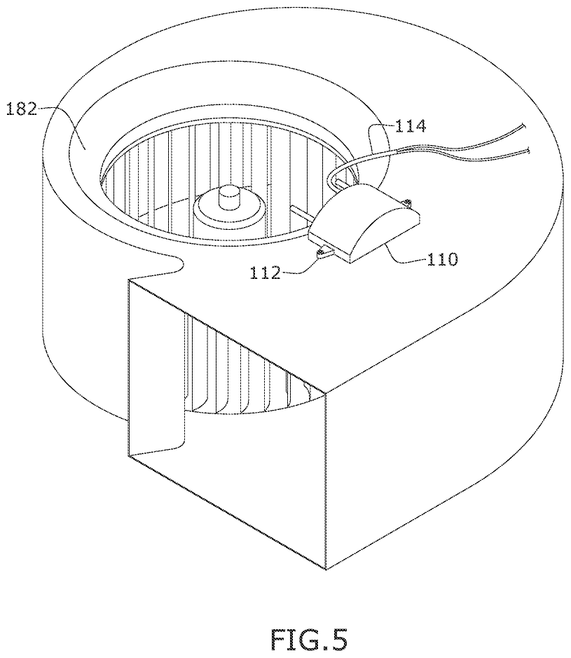

Referring now to the Figures, an exemplary embodiment of the present invention is described. FIG. 1 provides a perspective view of an exemplary embodiment of the present invention. FIG. 2 is an exploded view of an exemplary embodiment of the present invention. FIG. 3 is a perspective view of an exemplary embodiment of the present invention. FIG. 4 is an exploded schematic view of an exemplary embodiment of the present invention. FIG. 5 is a perspective view of an exemplary embodiment of the present invention.

As seen at FIGS. 1-5, an air treatment system in accordance with an exemplary embodiment of the present invention may include a commercial portable air conditioner 100 with a bipolar ionization assembly 110. The commercial portable air conditioner may be an ordinary commercial portable air conditioner found in the specialty industry. The commercial portable air conditioner may be a self-contained commercial portable air conditioner for cooling and heating a person, space or process.

As shown at FIG. 1, an air conditioning system may include an air conditioner with a bipolar ionization assembly. The air conditioner may be any suitable air conditioner. In some embodiments the air conditioner may be a commercially available portable air conditioner. In an exemplary embodiment the system may be a portable air conditioning system which includes a portable air conditioner unit with a bipolar ionization assembly.

The air conditioner may comprise a housing 120 for an evaporator or entry grill 130 for ambient air, condenser grill 140 for hot air discharge, and cold air supply discharge grate or nozzles 150 and an evaporator coil 166. Though not shown in the figures, the air conditioner may also include a refrigerant circuit compromising a compressor, and a condenser coil. Rotational castors or wheels 170 are affixed to the housing making it completely mobile in all aspects. A fan motor forces air through the evaporator coil 166 positioned within the housing chamber 120 to supply conditioned air into the space.

The air conditioning system further comprises a bipolar ionization assembly 110 positioned at the air stream of the intake of the evaporator side of the blower motor housing. A bipolar ionization assembly unit may be any suitable bipolar ionization assembly, which may be obtained from a commercial distributor or dealer. The commercial air conditioner or spot cooler would be disassembled, and the evaporator blower intake 182 would be located.

As shown at FIGS. 2-5, the bipolar ionizer assembly 110 may be attached by a bracket 112 with exact mounting specifications so that the electrical leads of the bipolar ionizer are placed within the air stream of the evaporator. After installing the bipolar ionization assembly, a power supply 114 may be connected from the commercial portable air conditioner to the bipolar ionization assembly. Once power is sourced to the assembly, the panels of the commercial portable air conditioner can be attached and ready for operation. All elements are necessary for the invention to work as a complete component.

The bipolar ionization assembly is attached to the blower intake of the evaporator housing. The bipolar ionization must be in the air stream of the evaporator section of the commercial portable air conditioner. It may be placed upstream or downstream within the airstream and still operate properly as intended.

The present invention incorporating a sanitizing bipolar ionization assembly within the portable air conditioner causes bipolar ionization of a gas creating plasma that is not in thermodynamic equilibrium because the ion temperature is lower than the electron temperature. This plasma is referred to as ‘cold plasma’ or ‘non-thermal plasma’ because it occurs at room temperatures. Cold plasma has many benefits which include, but are not limited to, the ability to kill harmful pathogens including bacteria, mycoplasma, viruses, mold and odors. Additionally, cold plasma helps with a reduction of Volatile Organic Compounds (VOC's) and a reduction of particulates in the air including known allergens. Furthermore, cold plasma also reduces or eliminates static electricity in the air.

The present invention may be used in numerous applications and industries. Currently, commercial portable air conditioners are sold and rented for short or long term cooling or heating applications. The present invention may be used when commercial HVAC systems break down or need maintenance. During such a downtime, the commercial portable air conditioner of the present invention may be used to cool buildings or facilities, nonlimiting examples of which include computer rooms, offices, classrooms, hospitals, health & rehabilitation facilities, medical facilities, and healthcare labs. The healthcare industry specifically uses commercial portable air conditioners within their facilities. Unlike existing alternatives, the present invention offers a safer product which provides sanitizing and elimination of odors, and dangerous microbes, viruses, bacteria and mold spores.

Nonlimiting examples of additional applications of the present invention may include food preparation, medical/pharmaceutical industries, automotive repair, construction and air remediation. For example, the present invention may be used in food preparation and processing to limit and eliminate germs while keeping food items cool. Uses in the medical and pharmaceutical industries may include sanitizing and sterilization of equipment. The present invention has practical applications in the automotive repair industry to eliminate toxic exhaust odors and VOCs in repair shops and automotive dealerships. The construction industry could also benefit from using the present invention by keeping job sites cooled and conditioned while removing odors from dangerous paint fumes and adhesives.

Further, the inventive commercial portable air conditioner with a bipolar ionization assembly has an important application as a remediation air scrubber, as an alternate air scrubber which may be used to remove particulate form the air. The remediation industry uses indoor air quality equipment such as HEPA air scrubbers. The present invention can be used in a similar fashion and produce better air quality than a HEPA air scrubber. The present invention distinguishes over all existing commercial portable air conditioners in indoor air quality, sanitization and purification.

In summary, the present invention provides an air treatment system comprising an air conditioner; a bipolar ionization assembly; and a power supply.

In some embodiments, the air conditioner is a portable air conditioning unit.

In some embodiments, the air conditioner is a self-contained commercial portable air conditioner for cooling and heating a person, space or process. The portable air conditioning unit comprises: an air conditioner housing; a plurality of wheels affixed to the housing; an evaporator or entry grill for ambient air; a condenser grill for hot air discharge; a cold air supply discharge grate; a motor; an evaporator coil; and an evaporator blower intake.

The air treatment system may further comprise a bracket constructed and arranged for mounting the bipolar ionizer assembly to the evaporator blower intake of the portable air conditioning unit. The bipolar ionization assembly further comprises a plurality of electrical leads. The bracket is constructed and arranged for positioning of the electrical leads of the bipolar ionizer within the air stream of the evaporator. The power supply is operably connected to the portable air conditioner and to the bipolar ionization assembly.

When mounted in the air conditioner, the bipolar ionization assembly is capable of causing bipolar ionization of a gas creating an antimicrobial plasma that is not in thermodynamic equilibrium. The antimicrobial plasma comprises ions having an ion temperature and electrons having an electron temperature, and the ion temperature is lower than the electron temperature. The bipolar ionization assembly is capable of causing bipolar ionization of a gas creating an antimicrobial plasma that is not in thermodynamic equilibrium because the ion temperature is lower than the electron temperature. The antimicrobial plasma is capable of killing harmful pathogens including bacteria, mycoplasma, viruses, mold and odors, and reducing the presence of Volatile Organic Compounds (VOC's), particulates, and static electricity in the air.

The present invention provides an air treatment method comprising the steps of providing an air treatment system as disclosed herein; and installing the bipolar ionization assembly in the portable air conditioner. The bipolar ionization assembly is installed in the portable air conditioner by disassembling the commercial portable air conditioner by locating and removing at least one access panel therefrom; locating the evaporator blower intake; providing a bracket attaching the bipolar ionization assembly to the blower intake of the evaporator housing so that the bipolar ionization assembly is located within the air stream of the evaporator; connecting a power supply from the commercial portable air conditioner to the bipolar ionization assembly; and reattaching the at least one access panel of the commercial portable air conditioner.

For clarity, only those aspects of the system germane to the invention are described, and product details well known in the art are omitted. In addition, many embodiments of the present invention have application to a wide range of industries. To the extent the is within the scope of the present invention. Further, to the extent the present application discloses a method, a system of apparatuses configured to implement the method are within the scope of the present invention.

It should be understood, of course, that the foregoing relates to exemplary embodiments of the invention and that modifications, variations and changes in detail may be made without departing from the spirit and scope of the invention as set forth in the following claims.

What is claimed is:

1. An air treatment system comprising:
an air conditioner;
a bipolar ionization assembly; and
a power supply.

2. The air treatment system of claim 1 wherein:
the air conditioner is a portable air conditioning unit.

3. The air treatment system of claim 2 wherein the air conditioner is a self-contained commercial portable air conditioner for cooling and heating a person, space or process.

4. The air treatment system of claim 2 wherein the portable air conditioning unit comprises:
an air conditioner housing;
a plurality of wheels affixed to the housing;
an evaporator or entry grill for ambient air;
a condenser grill for hot air discharge;
a cold air supply discharge grate;
a motor;
an evaporator coil; and
an evaporator blower intake.

5. The air treatment system of claim 4 further comprising a bracket constructed and arranged for mounting the bipolar ionizer assembly to the evaporator blower intake of the portable air conditioning unit.

6. The air treatment system of claim 5 wherein:
the bipolar ionization assembly further comprises a plurality of electrical leads;
the bracket is constructed and arranged for positioning of the electrical leads of the bipolar ionizer within the air stream of the evaporator; and
the power supply is operably connected to the portable air conditioner and to the bipolar ionization assembly.

7. The air treatment system of claim 6 wherein the bipolar ionization assembly when mounted within the portable air conditioner is capable of causing bipolar ionization of a gas creating an antimicrobial plasma that is not in thermodynamic equilibrium because the antimicrobial plasma comprises ions having an ion temperature and electrons having an electron temperature, and the ion temperature is lower than the electron temperature.

8. The air treatment system of claim 7 wherein the antimicrobial plasma is capable of killing harmful pathogens including bacteria, mycoplasma, viruses, mold and odors, and reducing the presence of Volatile Organic Compounds (VOC's), particulates, and static electricity in the air.

9. An air treatment method comprising the steps of:
a) providing an air treatment system as in claim 8; and
b) installing the bipolar ionization assembly in the portable air conditioner.

10. An air treatment method as in claim 9 wherein the step of installing the bipolar ionization assembly in the portable air conditioner further comprises:
disassembling the portable air conditioner by locating and removing at least one access panel therefrom;
locating the evaporator blower intake;
providing the bracket;
attaching the bipolar ionization assembly to the blower intake of the evaporator housing with the bracket so that the bipolar ionization assembly is located within the air stream of the evaporator;
connecting a power supply from the portable air conditioner to the bipolar ionization assembly; and
reattaching the at least one access panel of the portable air conditioner.
